(19) United States
(12) Patent Application Publication
(10) Pub. No.: US 2022/0071532 A1
(43) Pub. Date: Mar. 10, 2022

(54) BIOLOGICAL FLUID SAMPLING DEVICE

(71) Applicant: Neoteryx, LLC, Torrance, CA (US)

(72) Inventors: Stuart A. Kushon, Torrance, CA (US); Jeffrey Stonerook, Torrance, CA (US)

(21) Appl. No.: 17/017,554

(22) Filed: Sep. 10, 2020

(51) Int. Cl. A61B 5/15

(52) U.S. Cl. CPC A61B 5/150206; A61B 5/150022; A61B 5/150061

(57) ABSTRACT

A biological fluid sampling device includes a rigid body extending along a body axis and having a first end portion, a second end portion, and an intermediate portion extending between the first end portion and the second end portion. The second end portion includes a plurality of fins, with each fin extending radially outward relative to the body axis. Adjacent ones of the plurality of fins at least partially define a cavity therebetween. The device further includes an absorbent body connected to the rigid body and extending from the second end portion in a direction substantially parallel to the body axis, with the absorbent body being configured to absorb a biological fluid.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

### 1. Technical Field

The present disclosure relates generally to a biological fluid sampling device, and more specifically, to a device configured to preserve the integrity of the sampling material on the device as the device is manipulated and transferred during analysis thereof.

### 2. Description of the Related Art

Biological sampling and analysis are well known and extensively utilized to achieve many different objectives. For instance, blood samples are regularly used in clinical trials for pharmacokinetic analysis. Biological fluid samples are likewise typically needed for drug testing, crime scene investigations and patient analysis of various medical conditions and diagnoses. Field samples of various contaminated substances or unknown substances are collected for later laboratory analysis in a variety of applications.

To facilitate the collection of biological samples, elongate bodies having absorbent tips and corresponding containers for the elongate bodies have been developed. Exemplary bodies and containers are shown and described in United States Patent Application Publication No. 2013/0116597, entitled Method and Apparatus for Acquiring Blood for Testing, the contents of which are expressly incorporated herein by reference. Many conventional absorbent tips are positioned at an end of the elongate body and are placed in contact with the biological sample to allow the biological sample to be absorbed onto the tip. Once the biological sample has been absorbed, the elongate body is inserted into the container, with the absorbent tip passing through an opening formed in the container. The analysis of the biological sample may require subsequent withdrawal or removal of the elongate body from the container.

The configuration of many conventional sampling bodies and their corresponding absorbent tips may allow for inadvertent contact between the absorbent tip and the container, which may result in the absorbent tip being stripped off the elongate body. As such, the integrity of the sampling material may be comprised.

Another drawback with many conventional sampling bodies is that their configuration may occupy a significant percentage of volume in the container within which it is received. Oftentimes, the absorbent tips require bathing in an extraction solvent, and the large volume of the sampling body may prevent a less than desirable amount of extraction solvent within the container.

Accordingly, there is a need in the art for an improved sampling device which allows for insertion and subsequent removal of a sampling device into a container, while mitigating contact between the container and an absorbent material on the sampling device. Furthermore, there is a need for a sampling device having a reduced volume to allow for a greater volume of extraction solvent around the absorbent material. Various aspects of the present disclosure address these particular needs, as will be discussed in more detail below.

### BRIEF SUMMARY

Various aspects of the present disclosure are directed toward a sampling device having an end portion with a reduced volume to allow for increased levels of extraction solvent around the sampling device. Furthermore, additional aspects of the present disclosure are directed toward a sampling device configured to mitigate unwanted contact between an absorbent material on the sampling device and a container so as to preserve the integrity of the absorbent material.

In accordance with one embodiment of the present disclosure, there is provided a biological fluid sampling device comprising a rigid body extending along a body axis and having a first end portion, a second end portion, and an intermediate portion extending between the first end portion and the second end portion. The second end portion includes a plurality of fins, with each fin extending radially outward relative to the body axis. Adjacent ones of the plurality of fins at least partially define a cavity therebetween.

The device may additionally include an absorbent body engageable to the rigid body and configured to absorb a biological fluid.

The plurality of fins may include at least three fins. Each fin may extend away from the body axis and terminate at an outer edge, with the distance between the outer edge and the body axis defining a fin thickness that varies along the length of the fin. The outer edge may include a concave segment, a linear segment, and a tapered segment that extends from the linear segment toward the body axis.

The first end portion of the rigid body may include an end surface and an opening extending into the body from the end surface. The device may further comprise an internal surface extending from the end surface, with the internal surface being of a stepped configuration. The internal surface may include a cutout sized to receive an o-ring. The internal surface may include a first segment, a second segment, and a tapered segment extending between the first segment and the second segment, the tapered segment extending radially inward toward the body axis as the tapered segment extends from the first segment toward the second segment.

The second end portion may include a cylindrical body, with the plurality of fins extending between the cylindrical body and the intermediate portion.

The intermediate portion may include a cylindrical outer surface.

According to another embodiment, there is provided a biological fluid sampling device comprising a rigid body extending along a body axis and having a first end portion, a second end portion, and an intermediate portion extending between the first end portion and the second end portion. The second end portion includes a tapered profile relative to the intermediate portion. The biological fluid sampling device also includes an absorbent body engageable to the rigid body and configured to absorb a biological fluid.

The tapered profile may be defined by four tapered surfaces, each of which extend from the intermediate portion toward the absorbent body. The tapered profile may be defined by a domed surface.

The rigid body may include a hollow interior from the first end portion to the second end portion. The absorbent body may include a hollow interior in fluid communication with the hollow interior of the rigid body.

The intermediate portion may be of a circular, transverse cross section. The intermediate portion may be of a quadrangular, transverse cross section.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is an upper perspective view of a first embodiment of a biological fluid sampling device having a plurality of fins;

FIG. 2 is another upper perspective view of the first embodiment of the biological fluid sampling device taken from a different point of reference than that of FIG. 1;

FIG. 3 is a front view of the first embodiment of the biological fluid sampling device;

FIG. 4 is a bottom view of the first embodiment of the biological fluid sampling device having four fins;

FIG. 5 is a bottom view of a biological fluid sampling device having five fins;

FIG. 6 is an enlarged, partial, cross-sectional view of a first configuration of an internal profile of the biological fluid sampling device;

FIG. 7 is an enlarged, partial, cross-sectional view of a second configuration of an internal profile of the biological fluid sampling device;

FIG. 8 is an upper perspective view of a pair of the first embodiment biological fluid sampling devices in a transport cartridge;

FIG. 9 is a perspective view of the biological fluid sampling device connected to a sampling tool for collecting a biological sample;

FIG. 10 is an upper perspective view of the biological fluid sampling device connected to the sampling tool and being inserted into a holding rack;

FIG. 11 is a front view of a second embodiment of the biological fluid sampling device including a flow-through internal passageway;

FIG. 12 is a cross sectional view of the second embodiment of the biological fluid sampling device;

FIG. 13 is an upper perspective view of a third embodiment of the biological fluid sampling device including a square-shaped intermediate section and a four-sided, tapered end portion;

FIG. 14 is a front view of the third embodiment of the biological fluid sampling device;

FIG. 15 is a top view of the third embodiment of the biological fluid sampling device;

FIG. 16 is a bottom view of the third embodiment of the biological fluid sampling device;

FIG. 17 is a front view of a fourth embodiment of the biological fluid sampling device;

FIG. 18 is a front view of a sixth embodiment of the biological fluid sampling device;

FIG. 19 is a lower perspective view of a seventh embodiment of the biological fluid sampling device;

FIG. 20 is a side view of the seventh embodiment of the biological fluid sampling device; and

FIG. 21 is a bottom view of the seventh embodiment of the biological fluid sampling device.

Common reference numerals are used throughout the drawings and the detailed description to indicate the same elements.

### DETAILED DESCRIPTION

The detailed description set forth below in connection with the appended drawings is intended as a description of certain embodiments of a biological fluid sampling device and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

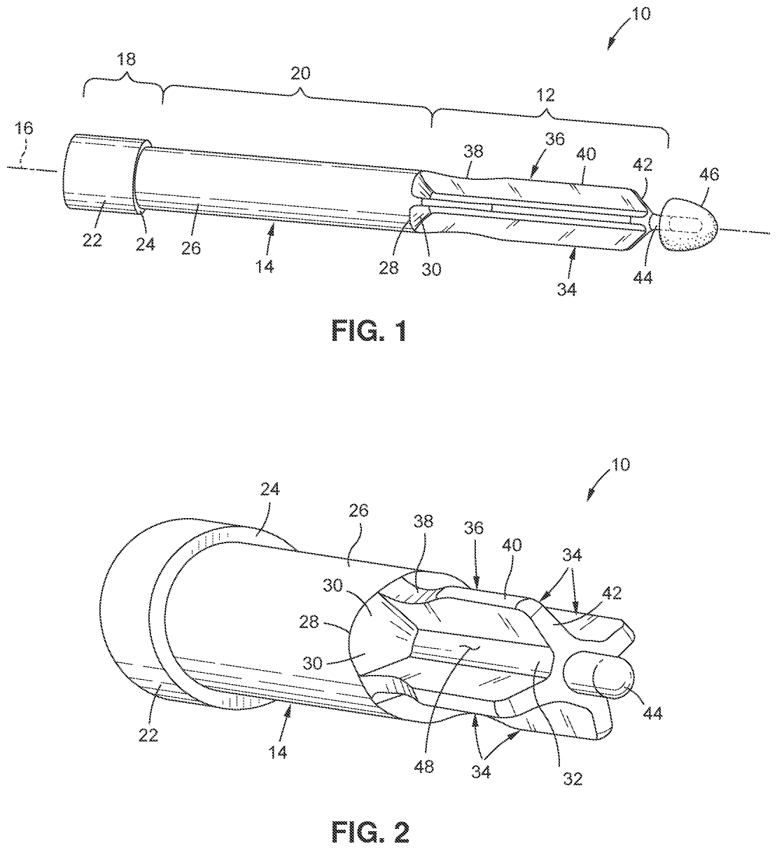

Referring now to FIGS. 1-4, there is depicted a first embodiment of a biological fluid sampling device 10 specifically configured and adapted to protect a biological sample collected by the sampling device 10 while insertion and removing the sampling device 10 from a holding rack. The sampling device 10 also includes a distal end portion 12 with a reduced volume to allow for more extraction solvent to surround the distal end portion 12, such as when the distal end portion 12 is received in a well plate, as is common when analyzing a biological fluid sample.

The sampling device 10 shown in FIGS. 1-4 includes a rigid body 14 extending longitudinally along a body axis 16 and having a first end portion 18 (e.g., proximal end portion), a second end portion 12 (e.g., distal end portion), and an intermediate portion 20 extending between the first end portion 18 and the second end portion 12. The first end portion 18 may be configured to interface with a sampling tool or other instrumentation that is selectively engageable with the sampling device 10, through a prescribed internal contour, as will be described in more detail below. The first end portion 18 may additionally include an outer surface 22 that is of a first diameter or circumference, that is larger than the immediately adjacent intermediate portion 20. As such, a shoulder 24 may be defined by the larger outer diameter of the first end portion 18, with the shoulder 24 extending between the outer surface 22 of the first end portion 18 and an outer surface 26 of the intermediate portion 20.

The intermediate portion 20 is positioned between the first and second end portions 18, 12 and may include the cylindrical outer surface 26 that is slightly smaller in diameter than the outer surface 22 of the first end portion 18. The intermediate portion 20 may be bounded on one end by the shoulder 24, and on the opposite end by an edge 28 at the interface of the intermediate portion 20 and an inwardly tapered surface 30 in the second end portion 12, so as to define an intermediate length between the two ends of the intermediate portion 20. The intermediate length may be sufficiently sized to allow for application of an adhesive label, sticker, or the like, which may be used to identify the patient, the sampling date, test to be performed, fluid extracted, test date, etc. In this regard, the intermediate portion 20 may include a generally constant, uniform diameter to provide a continuous surface for placement of the label, etc.

The second end portion 12 includes a base 32 and a plurality of fins 34 extending from the base 32 in a direction which is radially outward relative to the body axis 16. The fins 34 may be sized and configured to facilitate proper alignment with a hole or opening in a holding rack (see FIG. 10), which may be used to hold the sampling device 10 in a lab for testing the biological sample.

In order for the fins 34 to align the sampling device 10 relative to the hole or opening in the holding rack, the sampling device 10 may include at least three fins 34, and in some instances more than three fins 34. For example, the exemplary embodiment shown in FIGS. 1-4 includes four fins 34, while the embodiment shown in FIG. 5 includes five fins 34. It is contemplated that more than five fins 34 may be used without departing from the spirit and scope of the present disclosure. Irrespective of the number of fins 34 included on the sampling device 10, the spacing between the fins 34 may be equal. In this regard, if three fins 34 are used, the fins 34 may be spaced approximately 120 degrees relative to each other; if four fins 34 are used, the fins 34 may be spaced approximately 90 degrees relative to each other; if five fins 34 are used, the fins 34 may be spaced approximately 72 degrees relative to each other, and so forth.

Each fin 34 may extend away from the base 32 and terminate at an outer edge 36, with the distance between the outer edge 36 and the body axis 16 defining a fin thickness that may vary along the length of the fin 34. For instance, the outer edge 36 may include a concave segment 38, a linear segment 40, and a tapered segment 42 that extends from the linear segment 40 toward the body axis 16. The concave segment 38 may be positioned adjacent the intermediate portion 20, and transition into the linear segment 40, which extends away from the concave segment 38. The linear segment 40 may transition into the tapered segment 42, which may extend from the linear segment 40 toward the body axis 16. The plurality of linear segments 42 may converge at a tip 44, which extends away from the fins 34. The tip 44 may be configured to engage with an absorbent body 46, which may be selectively placed on the tip 44 for gathering a biological sample from a patient. The absorbent body 46 may be formed by an absorbent material, such as a porous material, a permeable material, or other materials known in the art. The absorbent body 46 may be sized and configured to absorb only a predetermined quantity of liquid, which may be less than about 100 μl, such as 10 μl, 20, μl, or 30 μl. Adhesives, ultrasonic bonding or other attachment mechanisms may be used to secure the absorbent body 46 onto the tip 44. The fluid that may be absorbed by the absorbent body 46 may include blood, saliva, serum, tears, cerebral spinal fluid, plasma, urine, synovial fluid, or other fluids known in the art.

According to one embodiment, the plurality of linear segments 40 may be the outward-most portion of the fins 34, and thus, collectively define a maximum outer diameter of the second end portion 12, which may be larger than the outer diameter of the intermediate portion 20. In other embodiments, the maximum outer diameter of the second end portion 12 may be substantially equal to, or less than, the outer diameter of the intermediate portion 20.

Adjacent fins 34 at least partially define a cavity 48 therebetween. The cavities 48 may be open ended adjacent the tip 44 and tapered at the opposite end due to the configuration of surface 30. The plurality of cavities 48 result in a reduction of volume of the sampling device 10, such that the reduced volume may be used to accommodate more extraction solvent around the second end portion 12 when the second end portion 12 is inserted into a well plate or other container including extraction solvent. The open end of the cavities 48 may additionally ease the insertion of the second end portion 12 into the extraction solvent without causing too much disruption (e.g., drag or turbulence) within the extraction solvent as the second end portion 12 is moved in the extraction solvent.

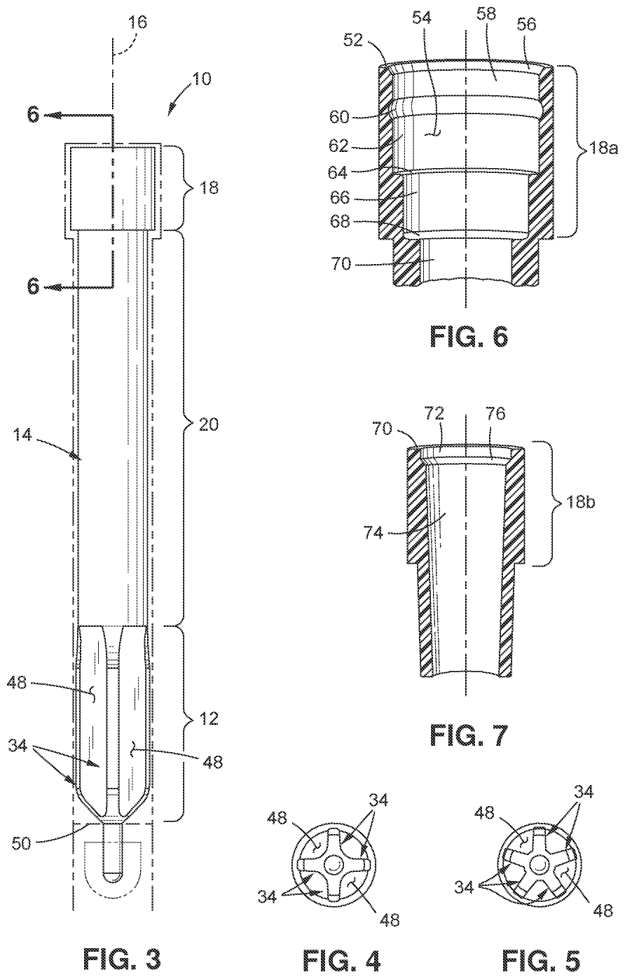

To illustrate the reduction in volume, FIG. 3 shows a cylindrical area 50 depicted in phantom, with the cylindrical area 50 being defined by the fins 34. In more detail, the top and bottom ends of the fins 34 define the ends of the cylindrical area 50, and the diameter defined by the fins 34 defines the diameter of the cylindrical area 50. The cavities 48 that extend between the fins 34 represent a void in the cylindrical area 50. The summation of these voids represents an enhancement in the volume available for additional extraction solvent. In other words, any volume that the sampling device 10 occupies in a well plate may reduce the amount of extraction solvent that can be used for extraction of the biological sample. Thus, the use of fins 34, and the intervening cavities 48, minimize the volume of the sampling device 10, which in turn, allows for an increase in the amount of extraction solvent that may be used.

Referring now to FIGS. 6 and 7, different hollow interiors of the sampling device 10 and their respective internal contours are depicted. The hollow interior and associated surface contours, particularly at the first end portion 18 thereof, may be specifically configured facilitate engagement with a sampling tool, a standard pipette, an automated liquid handler, or other devices known in the art. Furthermore, the hollow interior may also result in a reduction in total weight and manufacturing cost of the sampling device 10.

Referring specifically to FIG. 6, the first end portion 18a of the rigid body 14 may include an end surface 52 and an opening 54 extending into the rigid body 14 from the end surface 52. An internal surface may extend within the rigid body 14 from the end surface 52 toward the second end portion 12, with the internal surface having one or more steps formed therein. In the embodiment shown in FIG. 6, the internal surface includes a beveled portion 56 extending from the end surface 52, which then transitions into a first circular segment 58. An o-ring cutout 60 is formed between the first circular segment 58 and a second circular segment 62, with the o-ring cutout 60 being configured to receive an o-ring on a sampling tool to facilitate engagement between the sampling device 10 and the sampling tool. The second circular segment 62 may transition to a first internal shoulder 64, which then transitions to a third circular segment 66. A second internal shoulder 68 extends between the third circular segment 66 and a fourth circular segment 70. The configuration depicted in FIG. 6 may be used to facilitate engagement with liquid handler having a complementary configuration.

Referring now to FIG. 7, a different internal configuration of first end portion 18b is shown. In particular, the internal configuration depicted in FIG. 7 includes an end surface 70, a first circular segment 72, and a second circular segment 74 separated by a beveled or tapered segment 76. The second circular segment 74 may be tapered, such that the diameter thereof decreases as the distance from the end surface 70 increases. The configuration depicted in FIG. 7 may be particularly configured to facilitate engagement with handlers having a complementary configuration.

Although FIGS. 6 and 7 depict hollow internal configurations of the sampling device 10, it is contemplated that in other embodiments, the sampling device 10 may be solid, and thus, devoid of any internal hollowing.

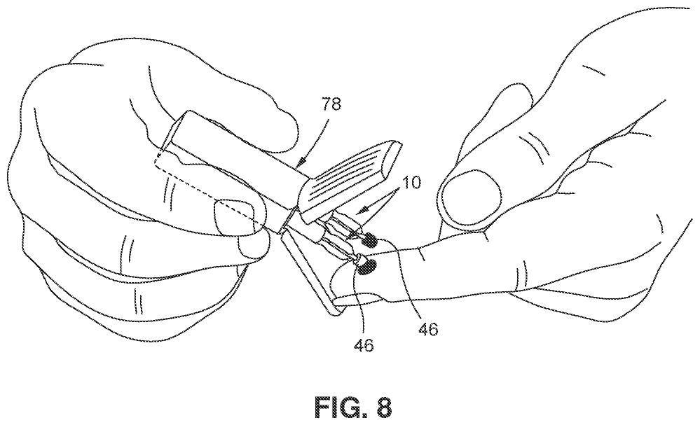

With the basic structure of the sampling device 10 having been described above, the following discussion focuses on exemplary uses of the sampling device 10. Referring first to FIG. 8, there is depicted a pair of sampling devices 10 in a transport cartridge 78. The sampling devices 10 are positioned to obtain a blood sample from a patient. As shown in FIG. 8, the absorbent body 46 on one of the sampling devices 10 has already absorbed blood, while the absorbent body 46 on the other sampling device 10 is in the process of absorbing blood. The configuration of the sampling devices 10 allows the sampling devices 10 to remain in the transport cartridge 78 when the sample is collected. Once the sample is collected, the transport cartridge 78 may be closed and shipped to a laboratory for testing.

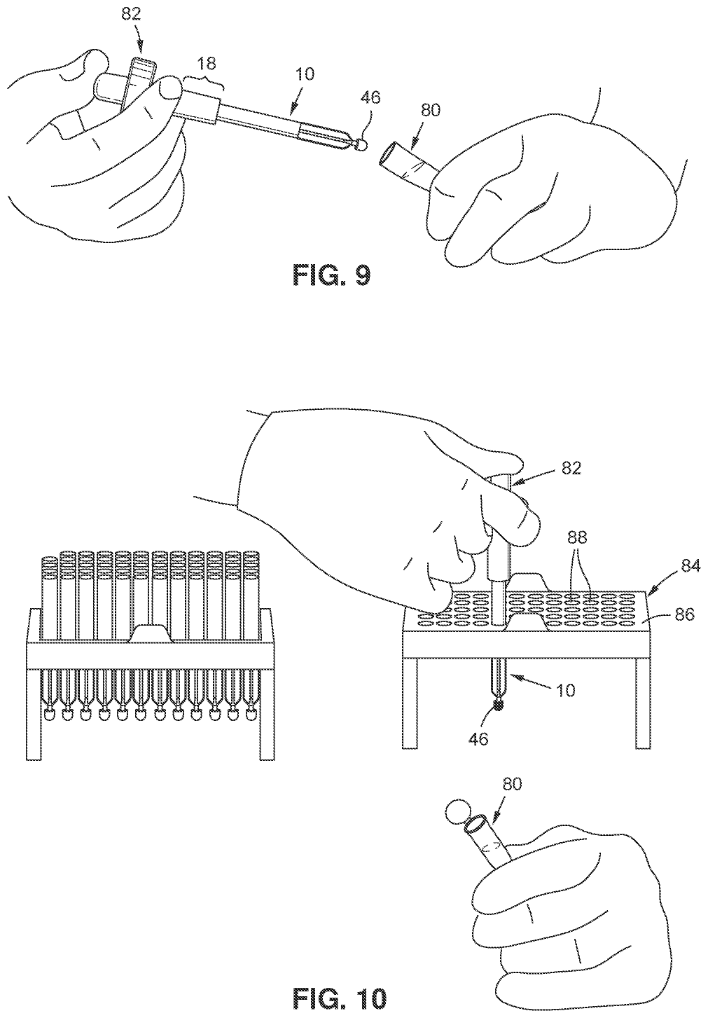

FIG. 9 is another view of a sampling device 10 used to collect a biological sample from a collection tube 80 by inserting the absorbent body 46 of the sampling device 10 into the biological fluid located in the collection tube 80. The sampling device 10 is connected to a sampling tool 82 to facilitate alignment and insertion of the sampling device 10 into the collection tube 80. In this regard, the first end portion 18 of the sampling device 10 may be adapted to receive, and engage with, a portion of the sampling tool 82. Once engaged, the user can control the positioning of the sampling device 10 while manually grasping the sampling tool 82.

After the biological sample has been collected, the sampling device 10 may be withdrawn from the collection tube 80 and placed in a holding rack 84, which includes an upper plate 86 with several openings 88 formed therein. For more information regarding an exemplary holding rack 84, please refer to U.S. Pat. No. 10,071,381, entitled Method and Apparatus for Handling Blood for Testing, the contents of which are expressly incorporated herein by reference.

As the sampling device 10 is inserted into its respective hole 88 on the holding rack 84, the fins 34 align the sampling device 10 with the hole 88 to facilitate proper insertion into the holding rack 84. Alignment of the sampling device 10 relative to the hole 88 may refer to the body axis 16 of the sampling device 10 being co-axial with a central axis about which the hole 88 is disposed. The intermediate portion 20 and/or the shoulder 24 between the first end portion 18 and the intermediate portion 20 may be wider than the hole 88 to allow the sampling device 10 to be supported by the holding rack 84 via abutment between the a portion of the rigid body 14 (e.g., the intermediate portion 20 or the shoulder 24) and the holding rack 84. When the sampling device 10 is in the holding rack 84, the second end portion 12 of the sampling device 10 may extend into a well plate (not shown), to bath the absorbent body 46 in an extraction solvent. The reduced volume created by the fin configuration allows for a greater volume of extraction solvent to surround the second end portion 12 of the sampling device 10.

The test procedure associated with the biological analysis may require removal of the sampling device 10 from the holding rack 84. When the sampling device 10 is retracted from its hole 88 on the holding rack 84, the configuration of the fins 34 ensures the sampling body 10 is withdrawn along the central axis of the hole 88, which prevents the absorbent body 46 from being inadvertently stripped off or otherwise removed from the rigid body 14. Thus, the absorbent body 46 may remain connected to the rigid body 14 to preserve the integrity of the absorbent body 46, and any test or analysis thereof.

Therefore, use of the sampling device 10 may require insertion and remove of the sampling device 10 from a hole on the holding rack 84. Accordingly, the size and configuration of the sampling device 10 may be specifically designed based on the size of the holes 88 in the holding rack 84.

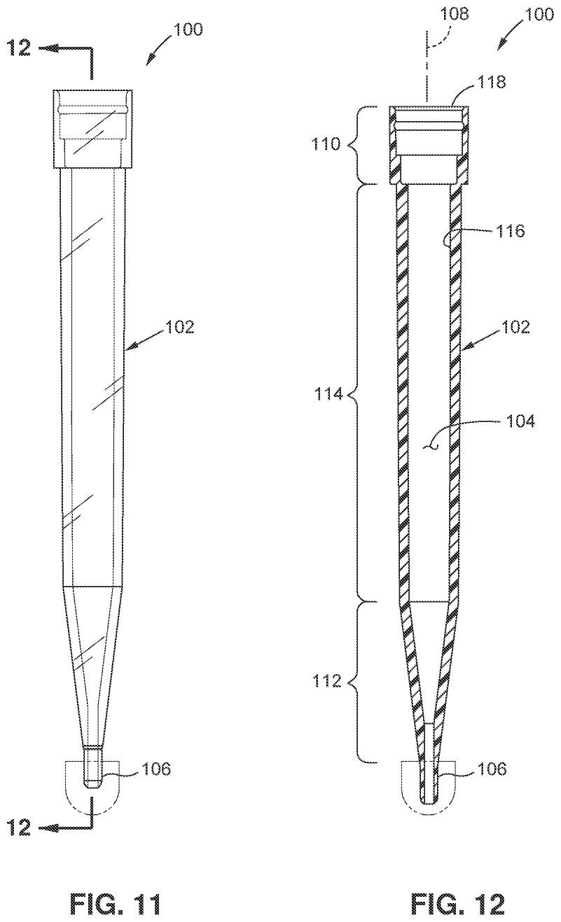

Referring now to FIGS. 11 and 12, there is depicted another embodiment of a sampling device 100 having a rigid body 102, which is hollow throughout a length thereof to provide a fluid passageway 104 therethrough. The fluid passageway 104 may be used for aspiration and/or dispensation of fluid via a tip 106.

According to such embodiment, the rigid body 102 extends along a body axis 108 and having a first end portion 110, a second end portion 112, and an intermediate portion 114 extending between the first and second end portions 110, 112. The external configuration of the first end portion 110 and intermediate portion 114 may be similar to that of the first embodiment described above; however, the second end portion 112 may have a distinct configuration. In particular, the outer surface of the second end portion 112 may be frustoconical in shape, with the outer diameter of the second end portion 112 decreasing from a maximum near the intermediate portion 114 to a minimum near the tip 106. The tapered configured of the second end portion 112 may result in a reduced volume of the second end portion 112 to enhance the volume available for an extraction solvent around the second end portion 112 when the second end portion is placed in a well plate, as described in more detail above.

An internal surface 116 of the rigid body 102 may extend around the body axis 108 from an end surface 118 of the first end portion 110 to an exposed end of the tip 106 so as to define the internal passageway 104. The internal passageway 104 adjacent the first end portion 110 may be configured to include an o-ring groove, a stepped configuration, or a stepped surface, to facilitate engagement with other tools, as described in more detail above.

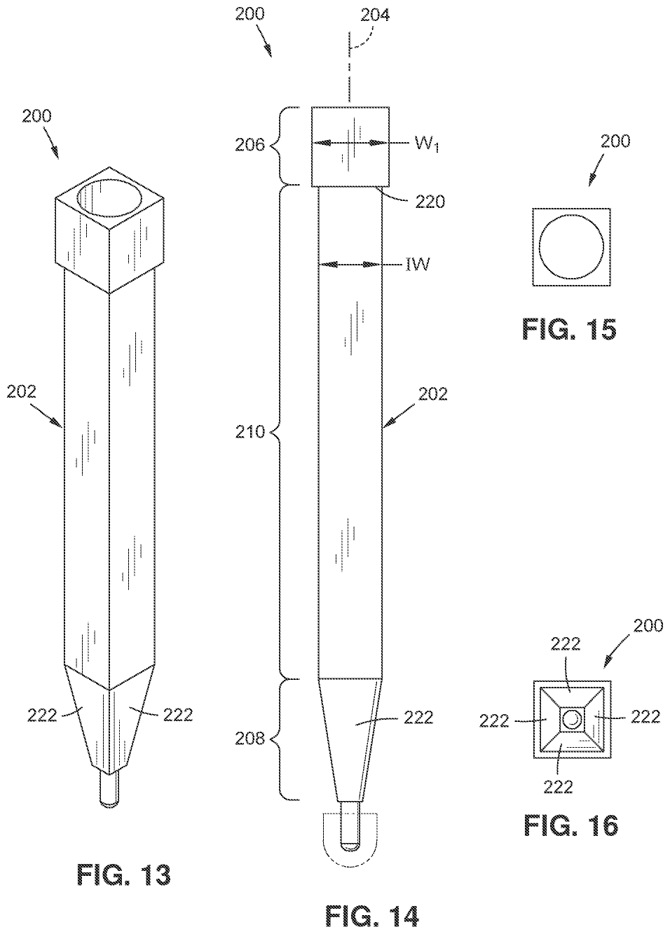

Referring now to FIGS. 13-16, there is depicted another embodiment of a sampling device 200 having a square-shaped, transverse cross-sectional configuration. The general configuration of the sampling device 200 includes a rigid body 202 extending along a body axis 204 and having a first end portion 206, a second end portion 208, and an intermediate portion 210 extending between the first and second end portions 206, 208. Each of the first end portion 206, second end portion 208 and intermediate portion 210 are configured to have a square cross-sectional configuration in a cross-sectional plane taken perpendicular to the body axis 204. The first end portion 206 defines a first width Wi as the distance between opposed outer surfaces thereof, and the intermediate portion defines an intermediate width IW as the distance between opposed outer surfaces thereof. The intermediate width IW may be less than the first width Wi, such that a shoulder extends between the intermediate portion 210 and the first end portion 206.

The second end portion 208 may be tapered in configuration, and include four planar surfaces 222, each of which extends from the intermediate portion 210 and is angled toward the body axis 204. More specifically, each of the planar surfaces 222 is angled toward an opposite planar surface 222.

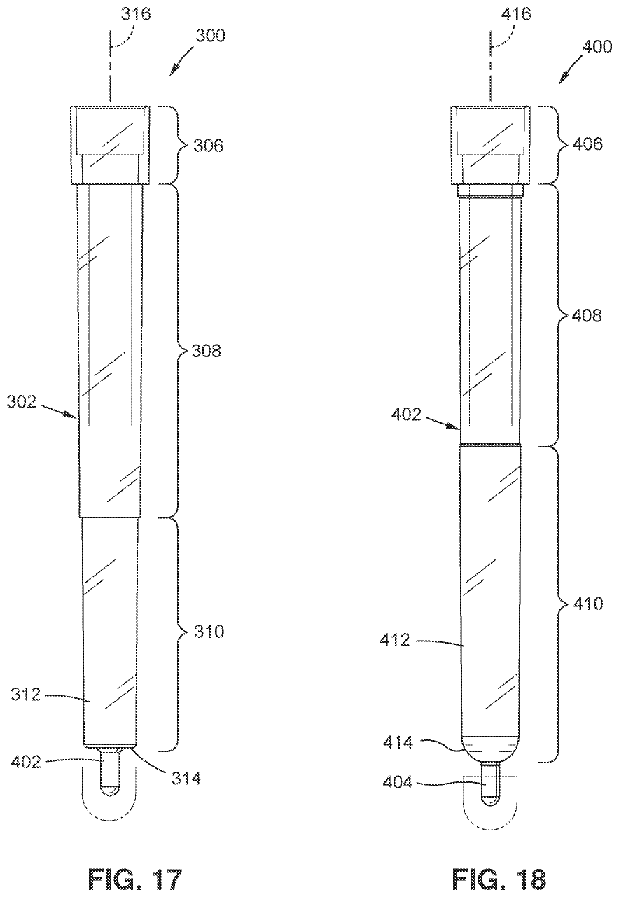

Referring now to FIG. 17, there is depicted another embodiment of a sampling device 300 including a rigid body 302 configured to include a broad nose adjacent a tip 304. In this respect, the rigid body 302 may include a first end portion 306 and an intermediate portion 308 that are similar in configuration to the embodiments described above. The primary distinction of the sampling device 300 of FIG. 17 relates to the second end portion 310, which is cylindrical in configuration and includes an outer surface 312 that is smaller in diameter than the outer surface diameter of the intermediate portion 308. The second end portion 310 may extend away from the intermediate portion 308 and terminate at an end surface 314, which may be generally perpendicular to a body axis 316. The second end portion 310 may include a beveled or tapered surface between the end surface 314 and the cylindrical outer surface 312. The tip 304 may extend from the end surface 314 and may be configured to engage with an absorbent body.

The configuration of the second end portion 310, with the cylindrical outer surface 312 having an outer diameter that is slightly less than the outer diameter of the intermediate portion 308, reduces the volume of the second end portion 310 to create more space for extraction solvent around the second end portion 310. However, the diameter of the second end portion 310 may be large enough to prevent inadvertent removal of an absorbent body positioned on the tip 304 as the sampling device 300 is withdrawn from a holding rack 84. In certain embodiments, the magnitude of the outer diameter of the second end portion 310 may be 65-95% of the magnitude of the outer diameter of the intermediate portion 308, more preferably, 70-90% of the magnitude of the outer diameter of the intermediate portion 308, and still more preferably, approximately 80% of the magnitude of the outer diameter of the intermediate portion 308.

FIG. 18 depicts yet another embodiment of a sampling device 400 including a rigid body 402 having a dome-shaped surface adjacent a tip 404. In this respect, the rigid body 402 may include a first end portion 406 and an intermediate portion 408 that are similar in configuration to the embodiments described above. The primary distinction of the sampling device 400 of FIG. 18 relates to the second end portion 410, which includes a cylindrical outer surface 412 that is substantially equal in diameter to the outer surface diameter of the intermediate portion 408. The cylindrical outer surface 412 may extend away from the intermediate portion 408 and transition into a dome-shaped surface 414, which may be disposed about a body axis 416. The dome-shaped surface 414 may be rounded, and in certain embodiments, semi-spherical. The tip 404 may extend from the dome-shaped surface 414 and may be configured to engage with an absorbent body.

The configuration of the second end portion 410, with the dome-shaped surface 414, reduces the volume of the second end portion 410, as compared to a squared off configuration, to create more space for extraction solvent around the second end portion 410. However, the diameter of the second end portion 410 may be large enough to prevent inadvertent removal of an absorbent body positioned on the tip 404 as the sampling device 400 is withdrawn from a holding rack 84.

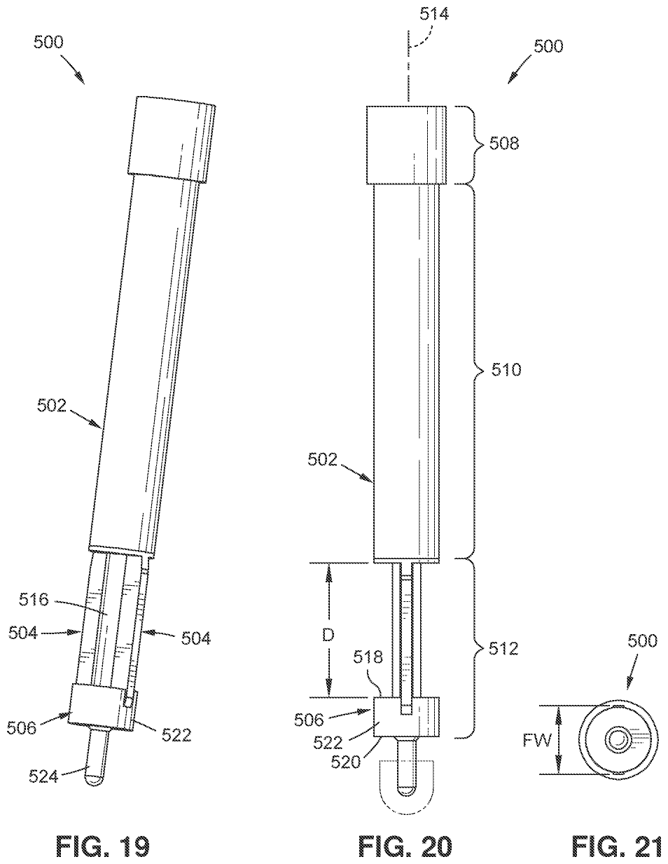

Referring now to FIGS. 19-21, there is depicted another embodiment of a sampling device 500 including a rigid body 502 configured to include a combination of a plurality of fins 504, as well as a cylindrical body 506. In this respect, the rigid body 502 may include a first end portion 508 and an intermediate portion 510 that are similar in configuration to the embodiments described above. The primary distinction of the sampling device of FIGS. 19-21 relates to the second end portion 512, which includes a pair of diametrically opposed fins 504 and the cylindrical body 506. In particular, the fins 504 extend longitudinally (e.g., in a direction parallel to a body axis 514) from the intermediate portion 510 to the cylindrical body 506. The fins 504 may also extend radially outward from a base surface 516 and terminate at an outer surface, with the opposed outer surfaces defining a fin width FW.

The cylindrical body 506 is spaced from the intermediate portion 510 by a distance, D, and includes an upper face 518 and a lower face 520 to define a cylindrical body height therebetween. A cylindrical outer surface 522 extends between the upper and lower faces 518, 520 and defines a cylindrical diameter.

In one embodiment, the outer diameter of the intermediate portion 510 is greater than the magnitude of the fin width FW. Furthermore, the fin width FW may be greater in magnitude than the cylindrical diameter. Thus, the magnitude of the physical features (e.g., outer diameter, fin width) may decrease from the intermediate portion 510 toward a tip 524.

The configuration of the fins 504 and cylindrical body 506 reduces the volume of the second end portion 512 to create more space for extraction solvent around the second end portion 512. The combination of the fins 504 and the cylindrical body 506, however, may be sufficient to prevent inadvertent removal of an absorbent body positioned on the tip 524 as the sampling device 500 is withdrawn from a holding rack 84.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A biological fluid sampling device comprising:
a rigid body extending along a body axis and having a first end portion, a second end portion, and an intermediate portion extending between the first end portion and the second end portion, the second end portion including a plurality of fins, each fin extending radially outward relative to the body axis, adjacent ones of the plurality of fins at least partially defining a cavity therebetween; and
an absorbent body engageable to the rigid body and being configured to absorb a biological fluid.

2. The biological fluid sampling device of claim 1, wherein the plurality of fins includes at least three fins.

3. The biological fluid sampling device of claim 1, wherein each fin extends away from the body axis and terminates at an outer edge, the distance between the outer edge and the body axis defining a fin thickness that varies along the length of the fin.

4. The biological fluid sampling device of claim 3, wherein the outer edge includes a concave segment, a linear segment, and a tapered segment that extends from the linear segment toward the body axis.

5. The biological fluid sampling device of claim 1, wherein the first end portion includes an end surface and an opening extending into the body from the end surface.

6. The biological fluid sampling device of claim 5, further comprising an internal surface extending from the end surface, the internal surface being of a stepped configuration.

7. The biological fluid sampling device of claim 6, wherein the internal surface includes a cutout sized to receive an o-ring.

8. The biological fluid sampling device of claim 6, wherein the internal surface includes a first segment, a second segment, and a tapered segment extending between the first segment and the second segment, the tapered segment extending radially inward toward the body axis as the tapered segment extends from the first segment toward the second segment.

9. The biological fluid sampling device of claim 1, wherein the second end portion includes a cylindrical body, with the plurality of fins extending between the cylindrical body and the intermediate portion.

10. The biological fluid sampling device of claim 1, wherein the intermediate portion includes a cylindrical outer surface.

11. The biological fluid sampling device of claim 1, wherein the second end portion includes a tip extending along the body axis, the absorbent body being engageable with the tip.

12. A biological fluid sampling device configured for use with an absorbent body, the biological fluid sampling device comprising:
a rigid body extending along a body axis and having a first end portion, a second end portion, and an intermediate portion extending between the first end portion and the second end portion, the second end portion including a plurality of fins and a tip extending along the body axis, each fin extending radially outward relative to the body axis, adjacent ones of the plurality of fins at least partially defining a cavity therebetween.

13. The biological fluid sampling device recited in claim 12, wherein each fin extends between the intermediate portion and the tip.

14. A biological fluid sampling device comprising:
a rigid body extending along a body axis and having a first end portion, a second end portion, and an intermediate portion extending between the first end portion and the second end portion, the second end portion including a tapered profile relative to the intermediate portion; and
an absorbent body engageable to the rigid body and configured to absorb a biological fluid.

15. The biological fluid sampling device recited in claim 14, wherein the tapered profile is defined by four tapered surfaces, each of which extend from the intermediate portion toward the absorbent body.

16. The biological fluid sampling device recited in claim 14, wherein the tapered profile is defined by a domed surface.

17. The biological fluid sampling device recited in claim 14, wherein the rigid body includes a hollow interior from the first end portion to the second end portion.

18. The biological fluid sampling device recited in claim 17, wherein the absorbent body includes a hollow interior in fluid communication with the hollow interior of the rigid body.

19. The biological fluid sampling device recited in claim 14, wherein the intermediate portion is of a circular, transverse cross section.

20. The biological fluid sampling device recited in claim 14, wherein the intermediate portion is of a quadrangular, transverse cross section.
